(19) United States
(12) Patent Application Publication
(10) Pub. No.: US 2018/0178033 A1
(43) Pub. Date: Jun. 28, 2018

(54) Method and Apparatus for LED Biostimulation in Ocular Tissue

(71) Applicants: John Taboada, San Antonio, TX (US); John Martin Taboada, San Antonio, TX (US)

(72) Inventors: John Taboada, San Antonio, TX (US); John Martin Taboada, San Antonio, TX (US)

(21) Appl. No.: 15/854,723

(22) Filed: Dec. 26, 2017

Related U.S. Application Data
(60) Provisional application No. 62/438,994, filed on Dec. 23, 2016.

(51) Int. Cl. A61N 5/06; F21V 23/02; F21V 3/00

(52) U.S. Cl. CPC A61N 5/0622; F21Y 2115/10; F21V 3/00; F21V 23/02

(57) ABSTRACT

A method and apparatus for biostimulation of ocular tissue are disclosed, including providing a tissue biostimulator, wherein the tissue biostimulator comprises: a power source; a housing; one or more LEDs positioned in the housing and powered by the power source; and one or more lenses configured to project an array of emitter plate images of the one or more LEDs onto the tissue; placing the tissue biostimulator up to an eye; powering on the tissue biostimulator; and projecting the array of emitter plate images of the one or more LEDs onto the tissue. Other embodiments are described and claimed.

### I. CROSS REFERENCE TO RELATED APPLICATIONS

This application claims the benefit of the filing date of U.S. Provisional Patent Application Ser. No. 62/438,994, filed on Dec. 23, 2016, entitled “Method and Apparatus for LED Biostimulation in Ocular Tissue,” the entire disclosure of which is hereby incorporated by reference into the present disclosure.

### II. BACKGROUND

There is a need for the treatment of retinal scotomas and other retinal wounds. A method and apparatus for LED biostimulation in ocular tissue for treating retinal scotomas and other retinal wounds is disclosed. The device may concurrently produce substantial biostimulation to a major portion of the whole eye.

### III. SUMMARY

In one respect, disclosed is a tissue biostimulator comprising: a power source; a housing; one or more LEDs positioned in the housing and powered by the power source; and one or more lenses configured to project an array of emitter plate images of the one or more LEDs onto the tissue.

In another respect, disclosed is a method for biostimulation of tissue, the method comprising: providing a tissue biostimulator, wherein the tissue biostimulator comprises: a power source; a housing; one or more LEDs positioned in the housing and powered by the power source; and one or more lenses configured to project an array of emitter plate images of the one or more LEDs onto the tissue; placing the tissue biostimulator up to an eye; powering on the tissue biostimulator; and projecting the array of emitter plate images of the one or more LEDs onto the tissue.

In yet another respect, disclosed is an ocular tissue biostimulator comprising: a power source; a housing; and an array of emitters having a single lens configured to project an array of stimulation points onto the tissue.

Numerous additional embodiments are also possible.

### IV. BRIEF DESCRIPTION OF THE DRAWINGS

Other objects and advantages of the invention may become apparent upon reading the detailed description and upon reference to the accompanying drawings.

FIG. 1 is a schematic diagram illustrating the LED characteristics, in accordance with some embodiments.

FIG. 2 is a schematic diagram illustrating an assembled array of LEDs, in accordance with some embodiments.

FIG. 3 is a schematic diagram illustrating an eye, shown in cross section, in close proximity to LED Assembly, in accordance with some embodiments.

FIG. 4 is a system block diagram, in accordance with some embodiments.

FIG. 5 is a photograph of an LED Assembly for biostimulation in ocular tissue, in accordance with some embodiments.

FIG. 6 is a flowchart illustrating a method for biostimulation of ocular tissue using the apparatuses illustrated in FIGS. 1 through 5 and FIG. 7, in accordance with some embodiments.

FIG. 7 is a photograph of an LED Assembly for biostimulation in ocular tissue, in accordance with some embodiments.

While the invention is subject to various modifications and alternative forms, specific embodiments thereof are shown by way of example in the drawings and the accompanying detailed description. It should be understood, however, that the drawings and detailed description are not intended to limit the invention to the particular embodiments. This disclosure is instead intended to cover all modifications, equivalents, and alternatives falling within the scope of the present invention as defined by the appended claims.

### V. DETAILED DESCRIPTION

One or more embodiments of the invention are described below. It should be noted that these and any other embodiments are exemplary and are intended to be illustrative of the invention rather than limiting. While the invention is widely applicable to different types of systems, it is impossible to include all of the possible embodiments and contexts of the invention in this disclosure. Upon reading this disclosure, many alternative embodiments of the present invention will be apparent to persons of ordinary skill in the art.

The biostimulation and wound healing characteristics of red light, approximately in the 600-700 nm range, is utilized in the present invention for the treatment and reduction of certain scotomas and other conditions in the retina, such as disease caused lesions or laser induced lesions from the laser treatment of retinal detachments.

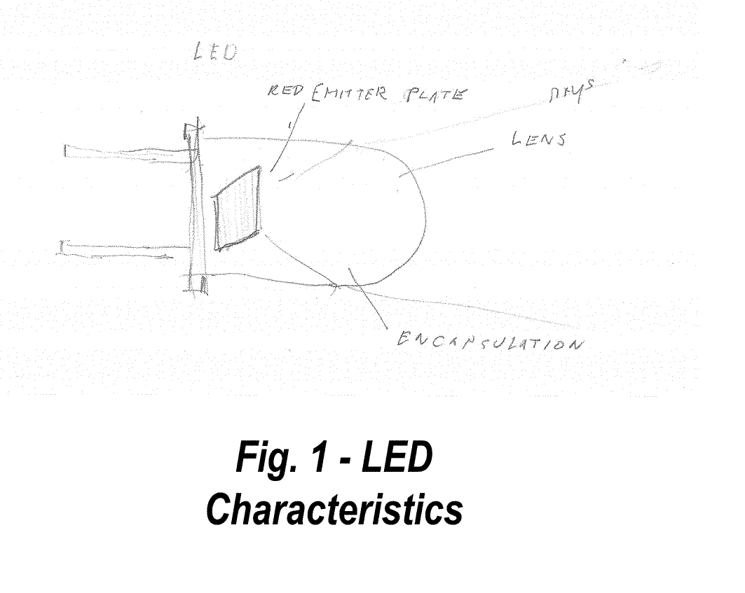

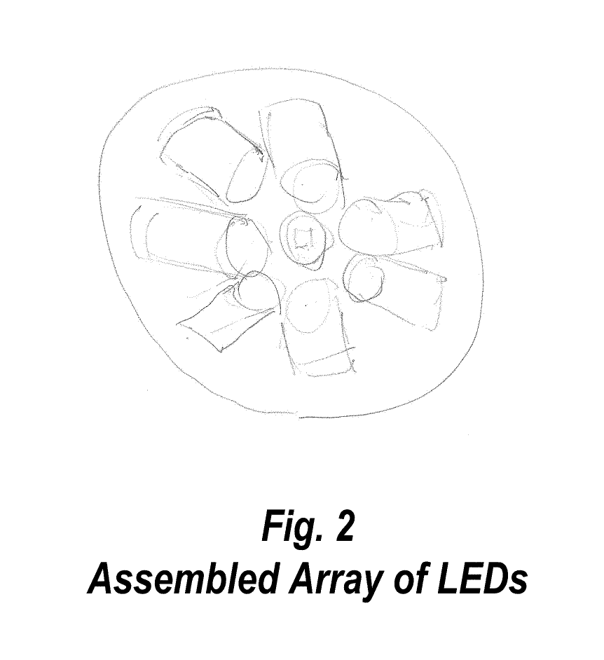

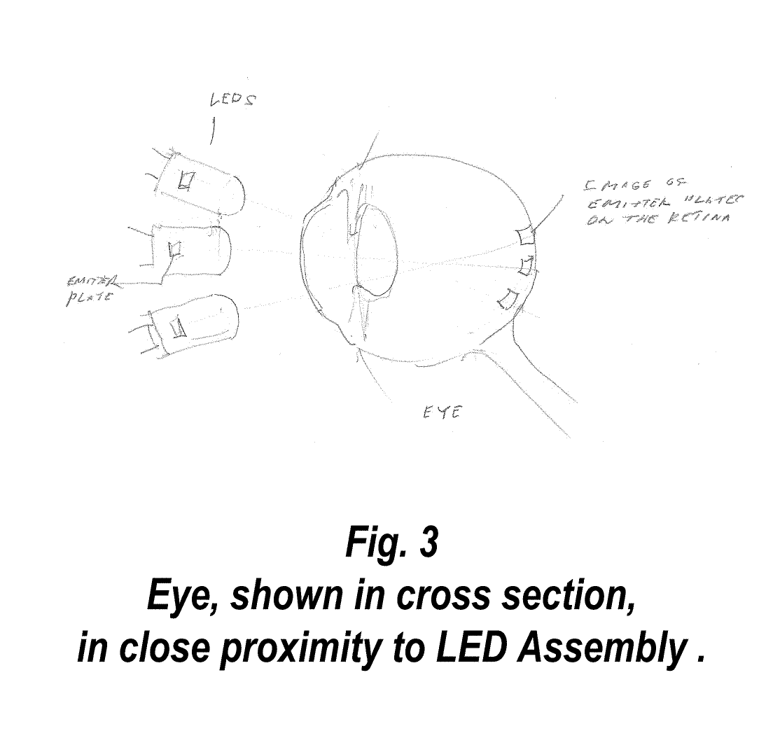

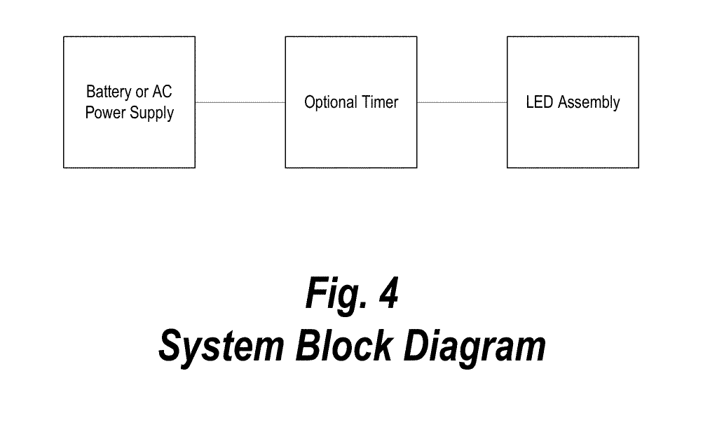

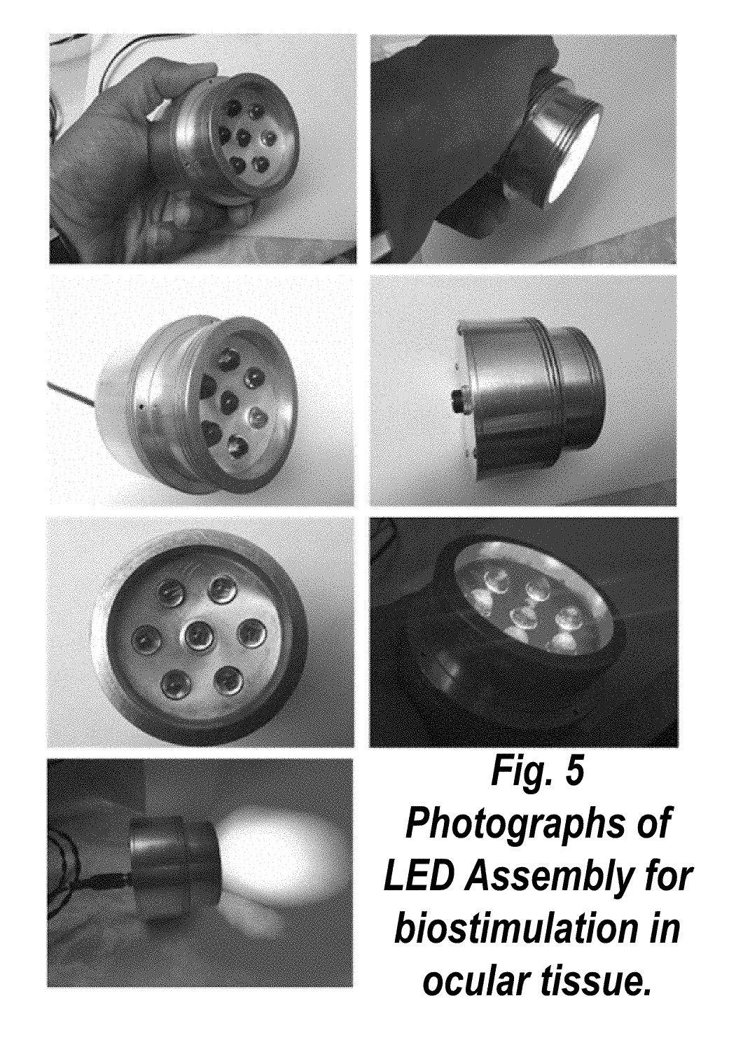

The red light emission may be obtained from one or more red emitting LED devices having a wavelength ranging from about 600 nm to about 700 nm and characterized by a square emitter plate encapsulated in a lens assembly as shown in FIG. 1. The LED devices may further be assembled into a tight focused array as shown in FIG. 2. When the assembly is placed in close proximity to the eye, the array of LED emitters project an array of emitter plate images. These images convey the healing treatment to the scotomas that may be present on the retina. FIG. 3 shows in cross section the function of the LED assembly. The LED plates are imaged in short range onto the retina, conveniently creating achievable therapeutic dose conditions on the retina. Other ocular tissue such as the cornea, aqueous, iris, trabecular meshwork, zonules, lens, and/or vitreous also receive biostimulation as they are in the path of the illumination for the LED devices. In some embodiments, the exposure is controlled by a timing circuit shown in the block diagram of FIG. 4. The proper red light exposure fluence multiplied by time of exposure is chosen to produce biostimulation of the wound healing processes. The treatment regimen may be delivered over a period of repeated doses as practiced in other wound healing or biostimulation practices. FIG. 5 are photographs of an LED Assembly, both in the off and on state, for biostimulation in ocular tissue in accordance with some embodiments.

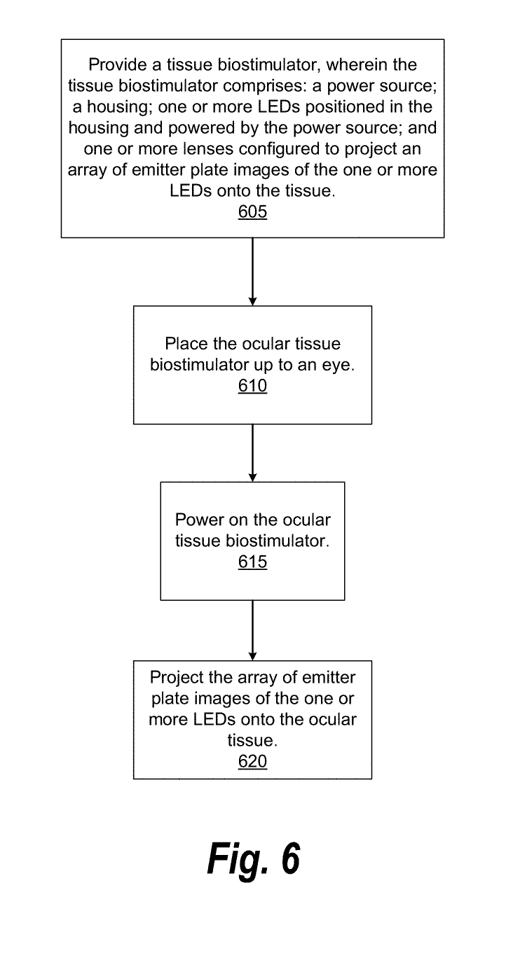

FIG. 6 is a flowchart illustrating a method for LED biostimulation in ocular tissue. According to one embodiment of the present invention, the method begins at block 605 where a tissue biostimulator is provided, wherein the tissue biostimulator comprises: a power source; a housing; one or more LEDs positioned in the housing and powered by the power source; and one or more lenses configured to project an array of emitter plate images of the one or more LEDs onto the tissue. At block 610, the tissue biostimulator is placed up to an eye. Next at block 615, the tissue biostimulator is powered on. At block 620, the array of emitter plate images of the one or more LEDs is projected onto the tissue.

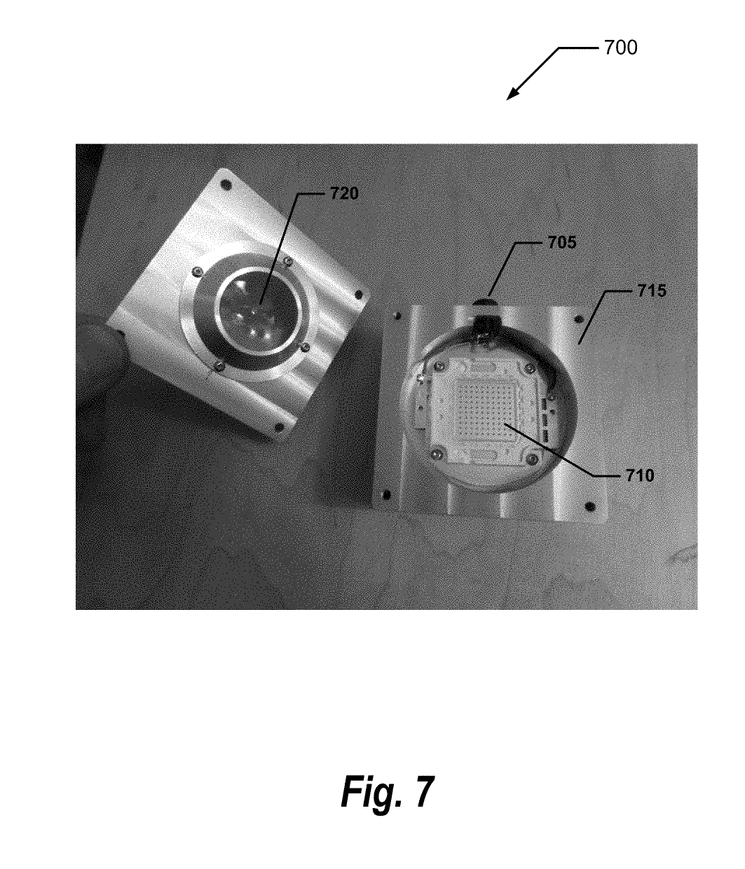

In the partially disassembled embodiment illustrated in FIG. 7, a tissue biostimulator comprises 700 a power source (not shown), a power connecter 705 for receiving the power source, a 10 by 10 array of LED emitters 710 mounted into a housing 715 and connected to the power source, and a single lens 720 configured to project an array of stimulation points onto the tissue. When fully assembled, the tissue biostimulator will project substantially similar to the tissue biostimulator photographed in FIG. 5.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

The benefits and advantages that may be provided by the present invention have been described above with regard to specific embodiments. These benefits and advantages, and any elements or limitations that may cause them to occur or to become more pronounced are not to be construed as critical, required, or essential features of any or all of the claims. As used herein, the terms “comprises,” “comprising,” or any other variations thereof, are intended to be interpreted as non-exclusively including the elements or limitations which follow those terms. Accordingly, a system, method, or other embodiment that comprises a set of elements is not limited to only those elements, and may include other elements not expressly listed or inherent to the claimed embodiment.

While the present invention has been described with reference to particular embodiments, it should be understood that the embodiments are illustrative and that the scope of the invention is not limited to these embodiments. Many variations, modifications, additions and improvements to the embodiments described above are possible. It is contemplated that these variations, modifications, additions and improvements fall within the scope of the invention as detailed within the following claims.

1. A tissue biostimulator comprising:
a power source;
a housing;
one or more LEDs positioned in the housing and powered by the power source; and
one or more lenses configured to project an array of emitter plate images of the one or more LEDs onto the tissue.

2. The tissue biostimulator of claim 1, wherein the tissue is a retina.

3. The tissue biostimulator of claim 1, wherein the one or more LEDs comprises a wavelength from about 600 nm to about 700 nm.

4. The tissue biostimulator of claim 1, further comprising a timer.

5. The tissue biostimulator of claim 1, wherein the tissue is a cornea, aqueous, iris, trabecular meshwork, lens, zonules, and/or vitreous.

6. A method for biostimulation of tissue, the method comprising:
providing a tissue biostimulator, wherein the tissue biostimulator comprises:
a power source;
a housing;
one or more LEDs positioned in the housing and powered by the power source; and
one or more lenses configured to project an array of emitter plate images of the one or more LEDs onto the tissue;
placing the tissue biostimulator up to an eye;
powering on the tissue biostimulator; and
projecting the array of emitter plate images of the one or more LEDs onto the tissue.

7. The method of claim 6, wherein the tissue is a retina.

8. The method of claim 6, wherein the one or more LEDs comprises a wavelength from about 600 nm to about 700 nm.

9. The method of claim 6, further comprising a timer.

10. The method of claim 6, wherein the tissue is a cornea, aqueous, iris, trabecular meshwork, lens, zonules, and/or vitreous.
